(19) United States
(12) Patent Application Publication
(10) Pub. No.: US 2018/0036092 A1
(43) Pub. Date: Feb. 8, 2018

(54) PACKAGE FOR A MEDICAL DEVICE

(71) Applicant: UFP Technologies, Inc., Georgetown, MA (US)

(72) Inventors: David R. Knowlton, Tewksbury, MA (US); Daniel J. Shaw, JR., Groveland, MA (US); Jeffrey Ryan Masters, San Diego, CA (US); Michael Joseph King, Topsfield, MA (US)

(21) Appl. No.: 15/788,101

(22) Filed: Oct. 19, 2017

Related U.S. Application Data
(63) Continuation-in-part of application No. 14/851,746, filed on Sep. 11, 2015.
Application No. 14/851,746 is a division of application No. 12/857,409, filed on Aug. 16, 2010, now Pat. No. 9,144,464.
(60) Provisional application No. 61/234,167, filed on Aug. 14, 2009.

(51) Int. Cl. A61B 50/30; A61F 2/00

(52) U.S. Cl. CPC A61B 50/30; A61F 2/0095; A61B 17/1222

(57) ABSTRACT

A package for a medical device includes a blister having a base and a plurality of walls extending from the base with the base and the walls defining a cavity for receiving the medical device. The blister includes a first thermoplastic polyurethane or a combination of the first thermoplastic polyurethane and polyethylene terephthalate glycol. The package also includes a lid disposed on the blister and at least partially covering the cavity. The lid has outer and inner surfaces and includes a high density polyethylene. The package further includes an insert disposed on and in direct contact with the inner surface of the lid and facing the cavity. The insert includes a second thermoplastic polyurethane for increasing puncture resistance of the lid from the medical device.

### CROSS-REFERENCE TO RELATED APPLICATION

This application is a Continuation-In-Part of U.S. patent application Ser. No. 14/851,746, filed on Sep. 11, 2015 which is a Divisional of U.S. patent application Ser. No. 12/857,409, filed Aug. 16, 2010, which claims priority to and all the advantages of U.S. Provisional Patent Application Ser. No. 61/234,167 filed Aug. 14, 2009, the disclosures of which are hereby expressly incorporated in their entirety by reference.

### FIELD OF THE INVENTION

The subject invention generally relates to a package for a medical device. More specifically, the subject invention relates to a package that includes a blister, a lid, and a thermoplastic polyurethane insert disposed on an inner surface of the lid to increase the puncture resistance of the lid from puncture from a medical device.

### DESCRIPTION OF THE RELATED ART

It is well known in the art to package medical devices in a variety of containers. These containers are typically designed to closely house the medical device. In other words, the medical device is typically packaged in very close contact with the containers to secure the device for shipping, sterilization, and use. Some containers include polyethylene cross-linked foam and glycol-modified polyethylene terephthalate (PETG) because these compounds tend to be clear, easily sterilized, and cost effective. However, many medical devices, such as those used to replace knee joints and hip joints, have one or more rough fixation surfaces. These rough surfaces typically have a coating formed from hydroxyl apatite. These rough surfaces tend to abrade the containers thereby creating undesirable shavings which contaminate the devices. Once abraded, the containers tend to lose rigidity and allow the devices to shift and move during shipping, sterilization, and use, all of which are undesirable. Furthermore, any coating on the rough surfaces can be rubbed off and removed.

In an attempt to minimize abrasion of the containers, and minimize removal of any coatings, efforts have been made to house medical devices in bags or pouches which are then disposed in the containers. Although the bags and pouches resist abrasion, their use increases production costs and processing complexities. In addition, the bags and pouches can be resistant to sterilization techniques which also increases costs, processing times, and overall suitability of use. Moreover, the bags and pouches do not provide non-slip surfaces to hold the devices in place in the containers. This tends to contribute to the shifting and moving of the devices in the containers during shipping, sterilization, and use. Accordingly, there remains an opportunity to develop an improved package for medical devices.

### SUMMARY OF THE INVENTION AND ADVANTAGES

The present invention provides a package for a medical device. The package includes a blister having a base and a plurality of walls extending from the base with the base and the walls defining a cavity for receiving the medical device. The blister includes a first thermoplastic polyurethane or a combination of the first thermoplastic polyurethane and polyethylene terephthalate glycol. The package also includes a lid disposed on the blister and at least partially covering the cavity. The lid has outer and inner surfaces and includes a high density polyethylene. The package further includes an insert disposed on and in direct contact with the inner surface of the lid and facing the cavity. The insert includes a second thermoplastic polyurethane for increasing puncture resistance of the lid from the medical device.

The present invention also provides a packaging system for packaging a medical device in a double sterile barrier. The packaging system includes a container including polyethylene terephthalate glycol and having a bottom and a plurality of walls extending from the bottom to define a void. The packaging system also includes a cover disposed on the container for at least partially covering the cavity and including a high density polyethylene. The packaging system further includes the aforementioned blister disposed within the void of the container.

### BRIEF DESCRIPTION OF THE SEVERAL VIEWS OF THE DRAWINGS

Other advantages of the present invention will be readily appreciated, as the same becomes better understood by reference to the following detailed description when considered in connection with the accompanying drawings wherein:

FIG. 1 is a top end perspective view of one embodiment of a composite;

FIG. 2 is a top side perspective view of another embodiment of a composite;

FIG. 3 is a top angled perspective view of yet another embodiment of a composite;

FIG. 4 is a bottom side perspective view of still another embodiment of a composite;

FIG. 5 is a bottom end perspective view of an additional embodiment of a composite;

FIG. 6 is a side cross-sectional view of one embodiment of a composite;

FIG. 7 is a side cross-sectional view of another embodiment of a composite;

FIG. 8 is a side perspective view of an outer container and a composite;

FIG. 9 is a side perspective view of another embodiment of a composite including a lid;

FIG. 10 illustrates one embodiment of an outer container that is further defined as a pouch with one open, but sealable, end.

FIG. 11 is a top end perspective view of one embodiment of a package for a medical device;

FIG. 12 is a top side perspective view of another embodiment of a package for a medical device;

FIG. 13 is a top angled perspective view of yet another embodiment of a package for a medical device;

FIG. 14 is a bottom side perspective view of still another embodiment of a package for a medical device;

FIG. 15 is a bottom end perspective view of an additional embodiment of a package for a medical device;

FIG. 16 is a side perspective view of one embodiment of a container, cover, blister, and lid used to form a double sterile barrier for a medical device;

FIG. 17 is a side perspective view of another embodiment of a package for a medical device including a lid;

FIG. 18 is a side cross-sectional view of another embodiment of a container, cover, blister, and lid of a package for a medical device.

FIG. 19 is a perspective view of an inner surface of a lid including an insert;

FIG. 20 is a bottom perspective view of a blister;

FIG. 21 is a top perspective view of a package for a medical device including a partially opened lid including an insert;

FIG. 22 is a top perspective view of another embodiment of a package for a medical device; and

FIG. 23 is an exploded view of a container, cover, blister, and lid used to form a double sterile barrier for a medical device.

### DETAILED DESCRIPTION OF THE INVENTION

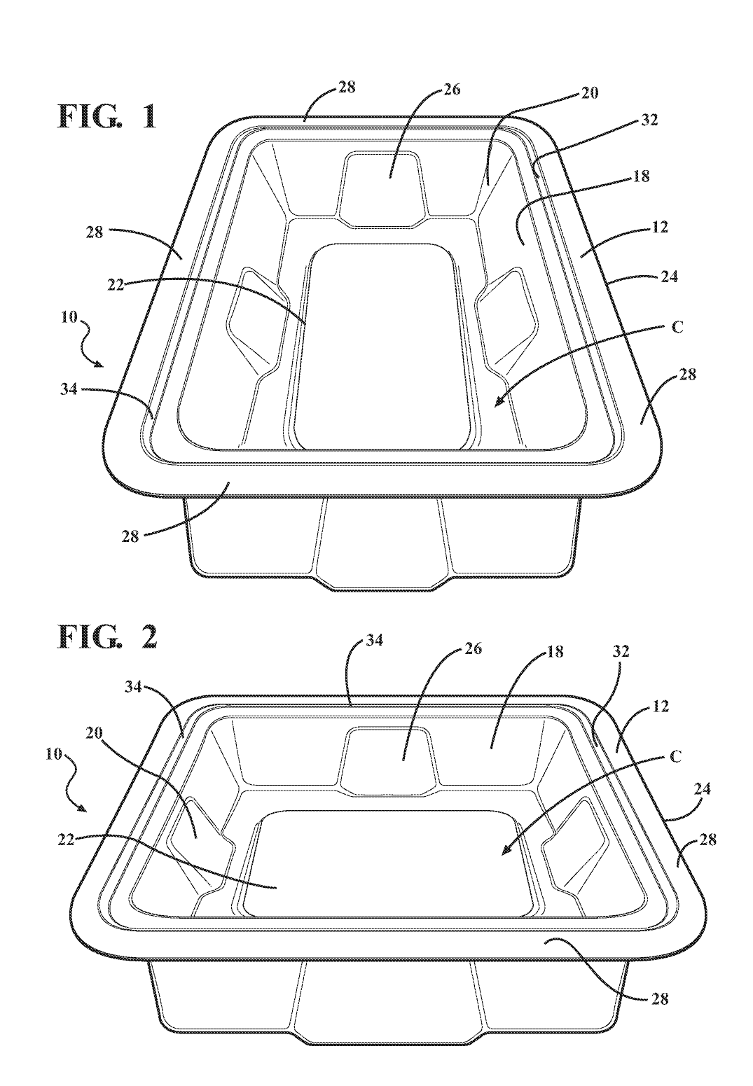

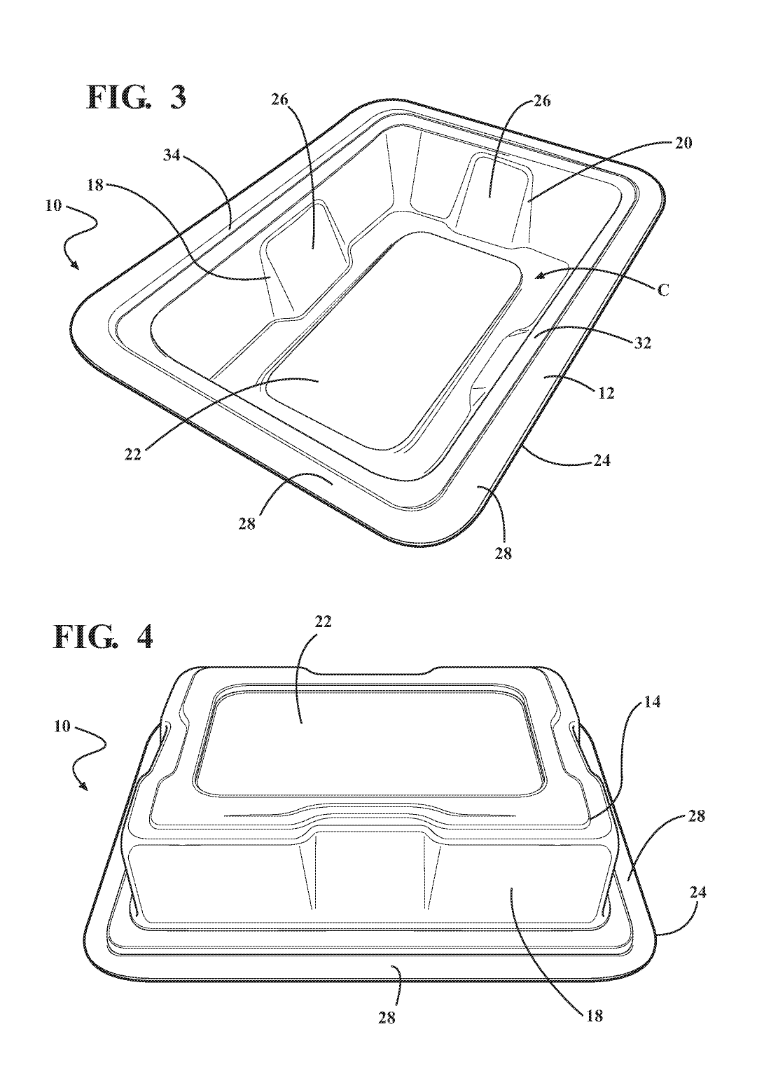

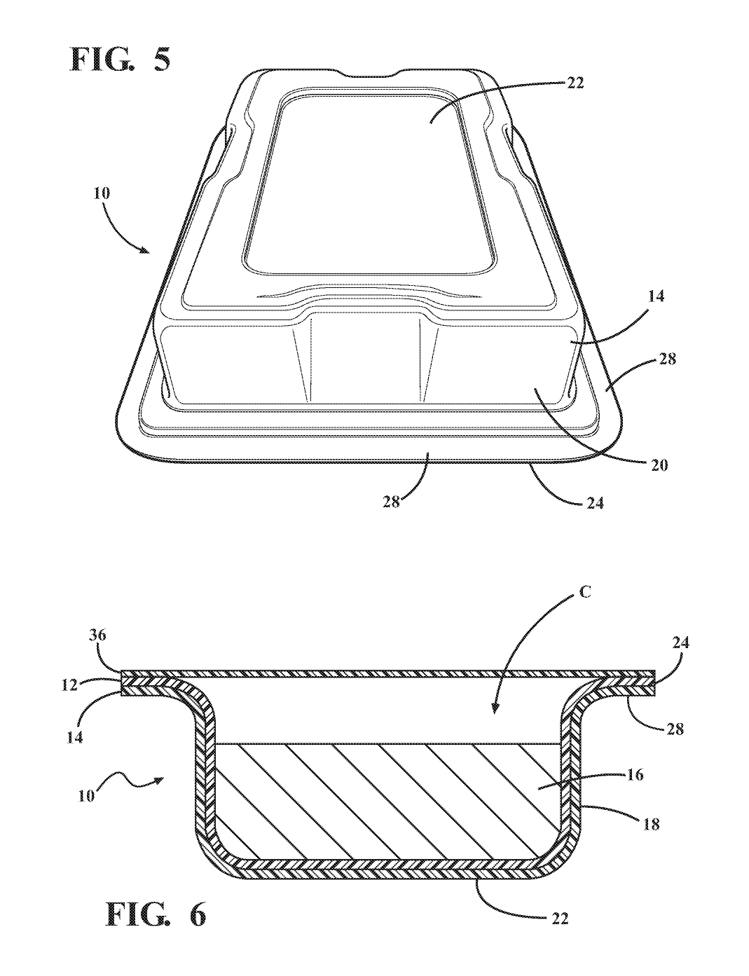

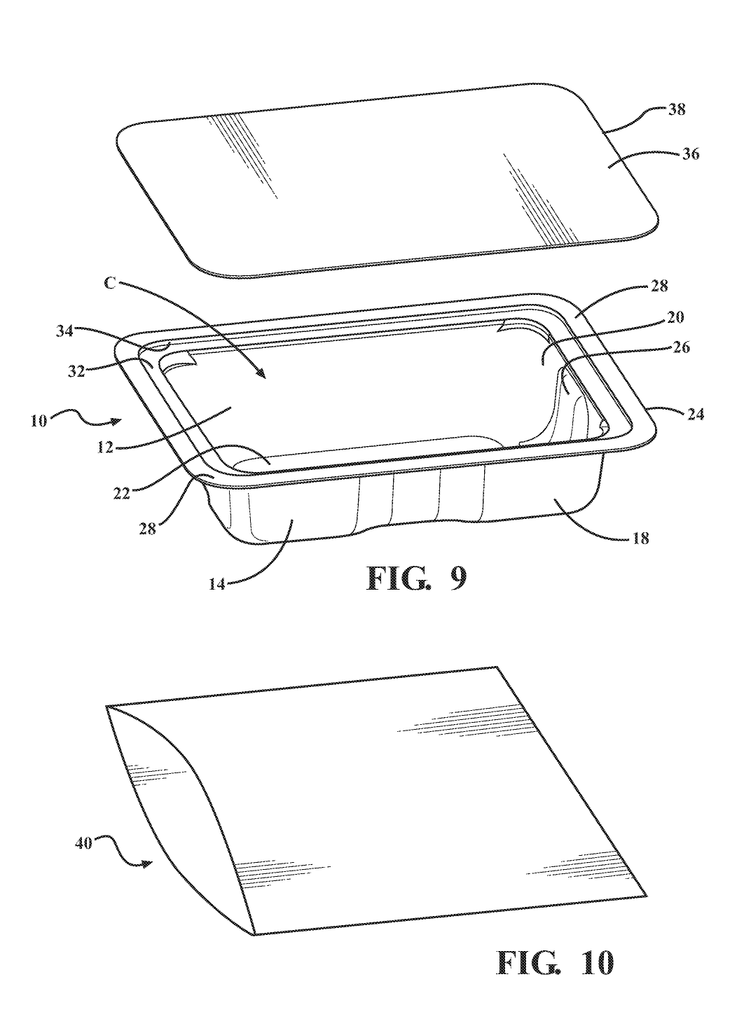

A composite (10) for packaging a medical device (16) is shown in FIGS. 1-8. The composite (10) may be further defined as a film, container, box, carton, bag, envelope, drum, can, bottle, or the like. In FIGS. 1-5 and 9, the composite (10) is further defined as a carton. In one embodiment, the composite (10) is further defined as a multi-layer film. In another embodiment, as set forth in FIG. 6, the composite (10) has two layers. The composite (10) may be sealed to itself or, alternatively, may be sealable to itself or sealable to an external substrate by any means known in the art including, but not limited to, manual sealing (e.g. with a fastener, clip, or string), thermal sealing, and/or chemical sealing (e.g. with melt-adhesion or chemical fusion).

The medical device (16) may be any known in the art and may have any shape and size. The medical device (16) may be, but is not limited to, a medical implant. Suitable examples of medical implants include, but are not limited to, hip implants, knee implants, ankle implants, dental implants, allografts, and the like. The medical device (16) may be formed from any composition including, but not limited to, metals, ceramics, biomaterials, apatite, and combinations thereof. Typically, the medical device (16) includes one or more surfaces. In one embodiment, these surfaces may be coated with hydroxyl apatite. These surfaces are commonly referred to in the art as “fixation surfaces.” In one embodiment, the medical device (16) is a hip implant. In another embodiment, the medical device (16) is a knee implant. In other embodiments, the medical device (16) is further defined as one of orthopedic (e.g. maxillofacial) implants, re-constructive prostheses, cardiac prostheses, joint implants, skin implants, dental implants, corneal implants, subcutaneous drug delivery devices such as medical implantable pills, drug-eluting stents, and the like. In yet other embodiments, the medical device (16) is further defined as a trauma device, surgical tool, plate, surgical fixation device, surgical accessory, or the like. The surgical fixation device may include, but is not limited to, screws, pins, and fasteners. It is contemplated that the medical device (16) may be simultaneously classified as more than one of the aforementioned types.

As shown in FIG. 6, the composite (10) includes a first layer (12) and a second layer (14). The first layer (12) is an innermost layer of the composite (10). In other words, the first layer (12) is an interior layer of the composite (10) that typically directly contacts the medical device (16). Of course, the composite (10) itself does not necessarily include the medical device (16) and the terminology “directly contacts the medical device (16)” refers to a design or ability of the first layer (12) to be in direct contact with the medical device (16) when the composite (10) is in use. However, in use, the composite (10) typically includes the medical device (16).

In various embodiments, the medical device (16) is at least partially disposed within a protective cover before being placed in the composite (10). The protective cover is not limited in composition, shape, or design. In one embodiment, the protective cover is further defined as a bag (e.g. a thermoplastic polyurethane bag) and the medical device (16) is placed inside of the bag. Alternatively, the protective cover may be further defined as a three dimensional foam in which the medical device (16) may be placed. In one embodiment, both a bag and a three dimensional foam are utilized. The medical device (16) and the protective cover may then be disposed within the composite (10) such that the first layer (12) of the composite (10) may be in direct contact with the protective cover.

The first layer (12) of the composite (10) includes an aromatic polyether polyurethane. Without intending to be bound by any particular theory, it is believed that the aromatic polyether polyurethane may create a non-slip surface that aids in cradling or restraining the medical device (16) in the composite (10). The aromatic polyether polyurethane is also typically abrasion resistant. The aromatic polyether polyurethane also, in part, provides protection to the composite (10) if the composite is dropped, is impacted, or is contacted with force. Typically, the aromatic polyether polyurethane is impact resistant and minimizes or prevents shattering, fragmentation, and/or breaking apart of the composite (10) upon impact. A particularly suitable aromatic polyether polyurethane is commercially available from Covestro under the trade name of Dureflex®. However, the composite (10) is not limited to use of this aromatic polyether polyurethane. The aromatic polyether polyurethane may be a rigid or flexible foam or may be an elastomer or a thermoplastic polyurethane. Alternatively, the first layer (12) may include a mixture of rigid foams, flexible foams, elastomers, and/or thermoplastic polyurethanes. In one embodiment, the aromatic polyether polyurethane is further defined as a film. In another embodiment, the aromatic polyether polyurethane is further defined as a sheet.

As is known in the art, aromatic polyether polyurethanes are typically formed from the reaction of a polyol and an isocyanate. For purposes of this invention, any suitable polyol and any suitable isocyanate may be used to form the aromatic polyether polyurethane. Typically, the polyol is further defined as a polyether polyol (i.e., a polyetherol). The polyether polyol may be aromatic or aliphatic. Alternatively, more than one polyol may be used to form the aromatic polyether polyurethane. A first polyol may be an aromatic polyether polyol and a second polyol may be an aliphatic polyether polyol. Similarly, the isocyanate may be further defined as an aromatic isocyanate or an aliphatic isocyanate. It is contemplated that more than one isocyanate may be used to form the aromatic polyether polyurethane. In one embodiment, an aromatic isocyanate and an aliphatic isocyanate are used to form the aromatic polyether polyurethane.

In various embodiments, the aromatic polyether polyurethane typically has the following physical properties, ±1%, 5%, 10%, 15%, 20%, or 25%, that are measured according to the described standardized tests. However, it is to be appreciated that the aromatic polyether polyurethane is not limited to the following physical properties and may include physical properties that are not described below or physical properties that may be different by more than ±1%, 5%, 10%, 15%, 20%, or 25% from those described below. Furthermore, the physical properties of the aromatic polyether polyurethane may be determined using standardized tests different from those described below.

| | |
|---|---|
| Specific Gravity | 1.12 |
| (ASTM D-792) | |
| Shore A Durometer Hardness | 87 |
| (ASTM D-2240) | |
| Ultimate Tensile Strength | 10,000 psi |
| (ASTM D-882) | |
| Ultimate Elongation | 575% |
| (ASTM D-882) | |
| 100% Modulus | 1150 psi |
| (ASTM D-882) | |
| 300% Modulus | 2700 psi |
| (ASTM D-882) | |
| Tear Resistance | 500 pli |
| (ASTM D-1004) | |
| Minimum Softening Point | 150° C. |
| (ASTM E2347-04) | |
| Maximum Softening Point | 170° C. |
| (ASTM E2347-04) | |
| Approximate Yield | 172 sq. ft/lb at 1 mil |

In one embodiment, the aromatic polyether polyurethane has a specific gravity of from 0.84 to 1.40 measured according to ASTM D-792.

In another embodiment, the aromatic polyether polyurethane a shore A hardness of from 66.25 measured according to ASTM D-2240 to a shore D hardness of 70.00.

In yet another embodiment, the aromatic polyether polyurethane has an ultimate tensile strength of from 7,500 to 12,500 psi, an ultimate elongation of from 431.25% to 718.75%, a 100% modulus of from 862.5 to 1437.5 psi, and a 300% modulus of from 2025 to 3375 psi, each measured according to ASTM D-882.

In still yet another embodiment, the aromatic polyether polyurethane has a tear resistance of from 375 to 625 pli measured according to ASTM D-1004.

In still yet another embodiment, the aromatic polyether polyurethane has a minimum softening point of from 112.5 to 187.5° C. and a maximum softening point of from 127.5 to 212.5° C. measured according to ASTM E2347-04.

In one embodiment, the first layer (12) includes the aromatic polyether polyurethane described above and a second polyurethane. The second polyurethane is not particularly limited any may be any polyurethane known in the art. The second polyurethane may be a second aromatic polyether polyurethane that is different from the aromatic polyether polyurethane first introduced above. In another embodiment, the second polyurethane is a polyester polyurethane. Alternatively, the first layer (12) may include the aromatic polyether polyurethane described above and one or more additional polyether and/or polyester polyurethanes.

In one embodiment, the first layer (12) consists essentially of the aromatic polyether polyurethane. In this embodiment, the terminology “consists essentially of” limits the first layer (12) from including any other polymer or compound that materially affects the basic and novel characteristics of the first layer (12). Typically, in this embodiment, the first layer (12) does not include any other polyurethanes, especially aromatic polyether polyurethanes. Alternatively, the first layer (12) may consist of the aromatic polyether polyurethane.

Typically, the first layer (12) is square or rectangular but may be of any shape and size. The first layer (12) typically has a thickness of from 0.5 to 250, more typically of from 1 to 60, still more typically of from 1 to 30, and most typically of from 2 to 30, mils. In other embodiments, the first layer (12) has a thickness of from 1 to 10 mils or from 2 to 10 mils. However, the instant composite (10) is not limited to these thicknesses. It is contemplated that the first layer (12) may have any thickness or range of thicknesses within the above ranges as determined by one of skill in the art. Typically, a thickness of the first layer (12) is correlated to a size, density, and/or weight of the medical device (16) to be used. However, the instant composite (10) is not limited to such a correlation. In one embodiment, the first layer (12) has a variable thickness of from 2 to 10 mils and may have segments of differing thicknesses. The first layer (12) may also be of any color, may be clear, opaque, or transparent. The first layer (12) may also be smooth or rough or have any texture known in the art.

Referring back to the second layer (14), the second layer (14) is disposed on the first layer (12), as shown in FIG. 6. Typically, no adhesive is disposed between the first and second layer (12, 14). In one embodiment, the second layer (14) is an outermost layer of the composite (10), i.e., forms at least a portion of an exterior of the composite (10) that is exposed to the environment. It is to be understood that the second layer (14) may be disposed in direct contact with the first layer (12) or may be disposed apart from the first layer (12) and still be disposed “on” the first layer (12). In one embodiment, the composite (10) includes more than two layers and the second layer (14) is an interior layer of the composite (10) (not shown in the Figures) that is not exposed to the environment.

The second layer (14) may be soft and flexible or may be rigid and stiff. Alternatively, the second layer (14) may include rigid and stiff segments while simultaneously including soft and flexible segments. The second layer (14) may be load bearing or non-load bearing and may be included in any portion of the composite (10). The second layer (14) may be a “top layer,” also known as a superstrate, or a “bottom layer”, also known as a substrate, of the composite (10). The second layer (14) typically functions as a load-bearing substrate that is an outermost layer of the composite (10). The second layer (14) also provides protection and impact resistance to the composite (10) but may crack or fragment upon impact. Typically, the second layer (14) is impact resistant and minimizes or prevents shattering, fragmentation, and/or breaking apart of the composite (10) upon impact. In one embodiment, the second layer (14) may crack but the aromatic polyether polyurethane (of the first layer (12)) remains intact and resists cracking to maintain the integrity (and sterility) of the composite (10).

The second layer (14) is not particularly limited in composition and may include one or more of a plastic, an organic polymer, an inorganic polymer, and combinations thereof. In various embodiments, the second layer (14) includes one or more of acrylics, polyesters, silicones, polyurethanes, halogenated plastics, polystyrenes, polyvinyl chlorides, polyethylene terephthalate glycols (PETG), polychlorotrifluoroethylenes (PCTFE), low-density polyethylenes, high-density polyethylenes, cross-linked, high-density polyethylenes, polyethylene foams, polycarbonates, polysulfones, fluorinated ethylene polypropylenes, ethylene-tetrafluoroethylenes, ethylene-chlorotrifluoroethylene copolymers, perfluoroalkoyl plastics, polypropylenes, cyclic olefin copolymers (COCs), and combinations thereof. In one embodiment, the second layer (14) includes PETG. In another embodiment, the second layer (14) includes a “breathable” foam, as known in the art.

In other embodiments, the second layer (14) consists essentially of an organic polymer, such as PETG. In this embodiment, the terminology “consists essentially of” limits the second layer (14) from including any other polymer or compound that materially affects the basic and novel characteristics of the second layer (14). Alternatively, the second layer (14) may consist of the organic polymer, e.g. consist of PETG.

The second layer (14) typically has a thickness of from 0.5 to 250, more typically of from 1 to 70, still more typically of from 1 to 60, and most typically of from 20 to 60, mils. In other embodiments, the second layer (14) has a thickness of from 10 to 70 mils or from 10 to 60 mils. However, the instant composite (10) is not limited to these thicknesses. It is contemplated that the second layer (14) may have any thickness or range of thicknesses within the above ranges as determined by one of skill in the art. In one embodiment, the second layer (14) has a variable thickness of from 20 to 60 mils. It is contemplated that the thickness of the second layer (14) may vary with the type of medical device (16) used with the instant composite (10). The second layer (14) may also be of any color, may be clear, opaque, or transparent. The second layer (14) may also be smooth or rough or have any texture known in the art.

It is also contemplated that the composite (10) may include one or more additional layers that are independent from the first and second layers (12, 14). The one or more additional layers may be the same or different from the first and second layers (12, 14) and are not limited in their orientation in the composite (10).

The first layer (12) forms a base (22) having a plurality of walls extending therefrom, typically including side walls (18) and end walls (20), and a periphery (24) (e.g. an edge) of the composite (10), as shown in FIGS. 1-5. The plurality of walls (e.g. side and/or end walls (18, 20)) may define one or more projections (26) which may engage the medical device (16) to retain its position in the composite (10). Typically, the side walls (18), end walls (20), and base (22) define a rectangular plan form but may define any shape plan form known in the art.

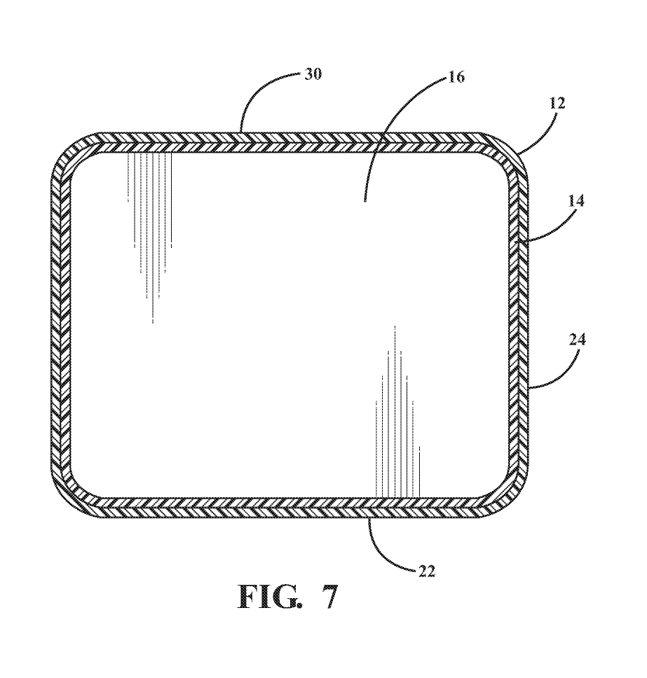

The plurality of walls (e.g. side walls (18) and end walls (20)) and the base (22) define a cavity (C). The cavity (C) may be open faced or entirely enclosed (i.e., defined on all sides (44)) by the side walls (18), end walls (20), and base (22). The second layer (14) is disposed on the first layer (12) opposite the cavity (C). The first and second layers (12, 14) may also form a top (30), as shown in FIG. 7, to entirely enclose the cavity (C). The cavity (C) may be defined as any shape including, but not limited to, cylindrical shapes, spherical shapes, conical shapes, rectangular shapes, cubic shapes, and the like. In one embodiment, the cavity (C) is defined as a shape that is the same as, substantially similar to, or complementary to, the shape of the medical device (16). In another embodiment, the cavity (C) is defined as a pocket. Typically the cavity receives the medical device.

The plurality of walls (e.g. side walls (18) and/or end walls (20)) may define top portions ending in flat or planar flanges (28) which are substantially parallel to the base (22). In one embodiment, a stepped recess (32) is formed in one or more of the flanges (28) and is disposed around the cavity (C). The stepped recess (32) typically includes a recess periphery (e.g. an edge of the stepped recess) (34) that is beveled upward to the flanges (28) which may support a lid (36).

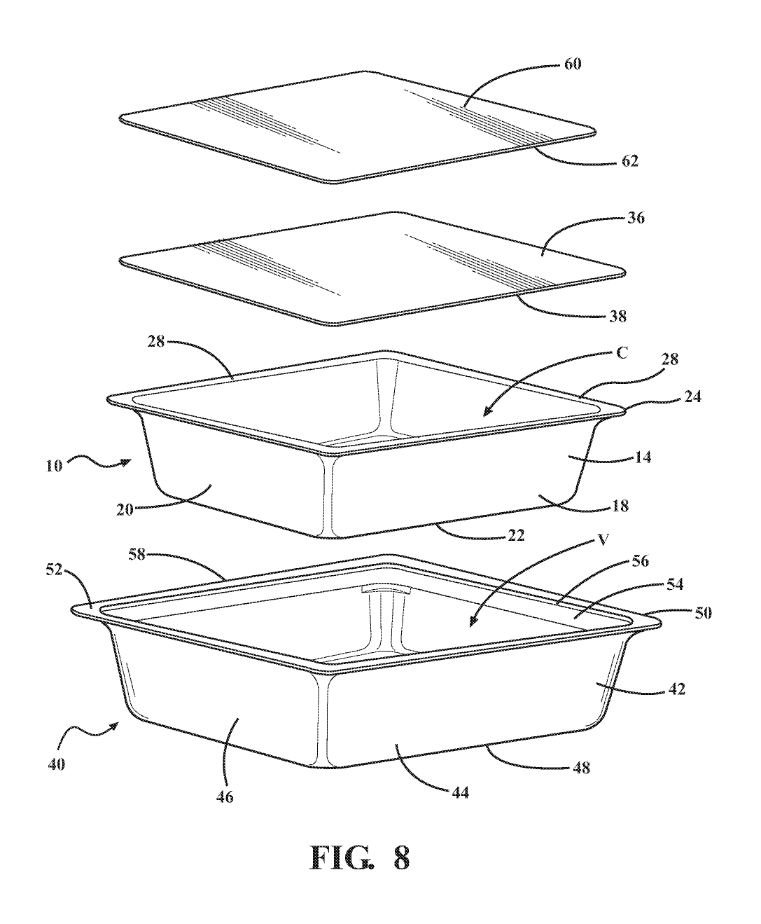

The lid (36) typically has a peripheral edge (38) and is typically disposed on the flanges (28), as shown in FIG. 8. The lid (36) may be sized and configured to cover the recess periphery (34) and the stepped recess (32) and the cavity (C) either in part or in their entirety. In one embodiment, the lid (36) is disposed such that the peripheral edge of the lid (38) aligns with the periphery (24) of the composite (10). In another embodiment, the lid (36) is disposed such that the peripheral edge of the lid (38) does not align with the periphery (24) of the composite (10). In this embodiment, the lid (36) is typically larger than or extends beyond the periphery (24) of the composite (10). The lid (36) is typically formed a suitable material in the art that can withstand sterilization with heat, chemicals, and/or radiation (e.g. ethylene oxide, gamma, e-beam, and/or peroxides). Suitable materials include, but are not limited to, a high density polyethylene such as Tyvek®, a combination of Tyvek® and polyethylene, Surlyn®, a combination of Surlyn® and Tyvek®, metal foils, polymer films, polyvinyl chloride (PVC), polychlorotrifluoroethylene (PCTFE), cyclic olefin copolymers (COCs), and combinations thereof. Typically, the lid (36) is formed from Tyvek®. Of course, the composite (10) is not limited to the aforementioned materials. The lid (36) typically has a thickness of from 1 to 60 mils but is not limited to this thickness. The lid (36) may have any thickness as chosen by one of skill in the art. The lid (36) may also be of any color, may be clear, opaque, or transparent. The lid (36) may also be smooth or rough or have any texture known in the art.

The lid (36) is typically adhered to the flanges (28) through use of an adhesive. The adhesive may be any known in the art and is not particularly limited. The adhesive may also have any thickness, as chosen by one of skill in the art.

Each of the layers of the composite (10) may independently and optionally include one or more conventional additives whose uses are well known to those skilled in the art. The use of such additives may be desirable in enhancing formation of the composite (10). Examples of such additives include oxidative and thermal stabilizers, impact modifiers such as thermoplastic olefins, thermoplastic elastomers, styrene butadiene rubber, lubricants, release agents, flame-retarding agents, oxidation inhibitors, oxidation scavengers, neutralizers, antiblock agents, dyes, pigments and other coloring agents, ultraviolet light absorbers and stabilizers, organic or inorganic fillers including particulate and fibrous fillers, reinforcing agents, nucleators, plasticizers, waxes, hot melt adhesives, biodegradation promoters, and combinations thereof. These additives may be used in any amount in any of the layers as determined by one of skill in the art.

In one embodiment, the composite (10) is further defined as a blister. As is known in the art, “blisters” or “blister packages” typically include a cavity defined by a polymer “web” and a lidding material that seals the cavity. The polymer “web” is typically formed by the first and second layers (12, 14) while the “lidding material” is typically defined as the lid (36). However, the instant composite (10) is not limited to these terms of art.

The composite (10) (e.g. the blister) is typically formed by a method that includes the step of disposing the first layer (12) on the second layer (14). The first layer (12) may be disposed on the second layer (14) by any means known in the art. Typically, the step of disposing the first layer (12) on the second layer (14) is further defined as thermoforming. Thermoforming typically includes any process involving heat, including but not limited to, laminating, radio frequency welding, ultrasonic welding, and co-extrusion. In another embodiment, the first layer (12) or the second layer (14) is die cut. However, the instant composite (10) is not limited to use of these techniques. Typically, thermoforming includes an automatic high speed positioning of a sheet or film of the composite (10) having an accurately controlled temperature into a pneumatically actuated forming station whereby a shape of the sheet or film is defined by a mold. The sheet or film, after positioned, may be trimmed or cut, as is known in the art. The thermoforming may utilize a variety of techniques including, but not limited to, use of a drape, vacuum, pressure, matched die, billow drape, vacuum snap-back, billow vacuum, plug assist vacuum, reverse draw with plug assist, trapped sheet, slip, diaphragm, twin-sheet cut sheet, and combinations thereof. The thermoforming may also utilize a freeblowing technique, a pressure bubble immersion technique, a twin-sheet roll-fed technique, a pillow-forming technique, a blow-molding technique, an extrusion blow-molding technique, and combinations thereof. Specifically, blow-molding includes expanding a heated parison against surfaces of a mold using compressed gasses. It is also contemplated that multiple blisters may be formed simultaneously using any of the aforementioned techniques, any technique known in the art, and combinations thereof.

In one embodiment, a film or sheet of the composite (10) is unwound from a reel and guided though a pre-heating station on a blister production line that utilizes upper and lower pre-heating plates. The temperature of the pre-heating plates is typically set such that the film or sheet of the composite (10) will warm, soften and become moldable. The warm film or sheet typically then arrives in a forming station where a large pressure (4 to 8 bar) forms the cavity into a negative mold. The negative mold is then cooled such that the film or sheet becomes rigid again and maintains its shape when removed from the mold. In some embodiments, the warm film or sheet is partially pushed down into the negative mold by a “plug-assist” feature.

In one embodiment, the method includes the step of disposing the one or more additional layers on the first and/or second layers (12, 14). The one or more additional layers may be disposed using any means known in the art. Typically, the one or more additional layers are also disposed using thermoforming, lamination, and/or co-extrusion techniques.

The blister may be of any desired shape and is typically formed in the shape of the medical device (16). In various embodiments, the blister is formed in rectangular or hemispherical shapes. After the blister is shaped, the medical device (16) is typically disposed within the blister, i.e. within the cavity, and the blister is preferably sealed with the lid (36). It is contemplated that the blister may define a space such that the medical device (16) is allowed to move within the blister. The blister may also include one or more spacers disposed therein to aid in holding the medical device (16) in place and/or to cushion the medical device (16). In one embodiment, the one or more spacers are further defined as foam spacers, e.g. cross-linked polyurethane foam spacers.

In various embodiments, the thickness of the first and second layers (12, 14) of the composite (10) varies at differing points on the blister or composite. Typically, these thicknesses are correlated to a depth of the cavity (C). For example, in blisters that have deep cavities (C), the thicknesses of the first and second layers (12, 14) may vary to a greater degree than in blisters that have shallow cavities (C). It is believed that the thicknesses of the first and second layers (12, 14) may also be correlated to a chosen method of formation, such as thermoforming. Of course, the instant composite (10) is not limited to these correlations. In various embodiments, the first and second layers have the following approximate gauges (inches):

| Gauge of First Layer (12) | Gauge of Second Layer (14) |
|---|---|
| 0.2500 | 0.2500 |
| 0.0250 | 0.0250 |
| 0.0200 | 0.0350 |
| 0.0150 | 0.0350 |
| 0.0200 | 0.0150 |
| 0.0150 | 0.0150 |
| 0.0010 | 0.0010 |
| 0.0005 | 0.0005 |

In other embodiments, one or more of these gauges may independently be different by more than ±1%, 5%, 10%, 15%, 20%, or 25%.

A packaging system for packaging the medical device (16) is also shown in FIG. 8. The packaging system typically includes the composite (10) (e.g. blister) disposed in one or more containers to form a double sterile barrier, e.g. a blister within a blister. The one or more containers may be further defined as films, boxes, cartons, bags, envelopes, drums, cans, bottles, or the like. The one or more containers may be formed from any suitable material known in the art including, but not limited to organic polymers such as acrylics, polyesters, silicones, polyurethanes, halogenated plastics, polystyrene, polyvinylchloride, polyethylene terephthalateglycol (PETG), polychlorotrifluoroethylene (PCTFE), low-density polyethylenes, high-density polyethylenes, cross-linked, high-density polyethylenes, polycarbonates, polysulfones, fluorinated ethylene polypropylene, ethylene-tetrafluoroethylene, ethylene-chlorotrifluoroethylene copolymer, perfluoroalkoyl, polypropylene, and combinations thereof. In one embodiment, the one or more containers include PETG. In various other embodiments, the one or more containers may include Tyvek®, a combination of Tyvek® and polyethylene, Surlyn®, or a combination of Surlyn® and Tyvek®.

The one or more containers may be identical to the composite (10) or may be different, e.g., in an embodiment where the one or more containers is identical to the composite (10), the one or more containers include a first container layer and a second container layer identical to the first layer (12) and the second layer (14), respectively, of the composite (10) described in greater detail above. In this embodiment, the first container layer forms a floor (48) having a plurality of container walls extending therefrom, typically including sides (44) and ends (46). The sides and/or ends (44, 46) define a void (V) for receiving the composite (10) and the second container layer is disposed on said first container layer opposite said void (V).

In one embodiment, as set forth in FIG. 8, the one or more containers is further defined as an outer container (40) that surrounds the composite (10) (e.g. blister). In other words, the composite (10) is disposed within the outer container (40). Said differently, the outer container (40) houses the composite (10). In this embodiment, the outer container (40) is exposed to the environment. In one embodiment, the outer container (40) is further defined as a pouch with one open, but sealable, end, as illustrated in FIG. 10. The open end may be sealed by any means known in the art including physical and/or chemical means. In one embodiment, the end is sealed to create a sterile barrier. In another embodiment, the end is not sealed.

The one or more containers (e.g. the outer container (40)) typically include a body (42) that is integrally formed with a plurality of container walls, including sides (44) and ends (46), and a floor (48), which together define a void (V) in which the composite (10) may be disposed. The sides (44) and/or ends (46) may define top portions (50) ending in flat or planar surfaces (52) that are substantially parallel to the floor (48). In one embodiment, a stepped indentation (54) is formed in one or more of the surfaces and is disposed around the void (V). The stepped indentation (54) typically includes an edge (56) that is beveled upward to the flat or planar surfaces (52). The container also defines a periphery (58).

The container may also include a cover (60) that includes a peripheral edge (62). The cover (60) may be the same as the lid (36) or may be different. Typically, the cover (60) is disposed such that the peripheral edge of the cover (62) aligns with a periphery of the container (58). The cover (60) may be formed from any suitable material in the art that can withstand sterilization with heat, chemicals and/or radiation (e.g. ethylene oxide, gamma, e-beam, and/or peroxides). Suitable materials include, but are not limited to, Tyvek®, metal foils, polymer films, a combination of Tyvek® and polyethylene, Surlyn®, a combination of Surlyn® and Tyvek®, and combinations thereof. Typically, the cover (60) is formed from Tyvek®. The cover (60) typically has a thickness of from 1 to 60 mils but is not limited to this thickness. The cover (60) may have any thickness as chosen by one of skill in the art. The cover (60) may also be of any color, may be clear, opaque, or transparent. The cover (60) may also be smooth or rough or have any texture known in the art.

A packaging system for packaging the medical device (16) in the container is also described. In one embodiment the packaging system includes the container for receiving the medical device (16) and the composite (10). The composite (10) is disposed in the container and contacting the medical device (16). Typically, the composite (10) supports the medical device (16) disposed in the container. In this embodiment, the composite (10) can be defined as a support for the medical device (16) having any suitable configuration. Typically, the composite (10) cradles the medical device (16) and is complementary in shape to the medical device (16).

Both the one or more containers, and the packaging system itself, may be formed by any method or means known in the art. Typically, the one or more containers are formed using thermoforming and/or lamination techniques. However, the instant invention is not limited to such processes. The packaging system is typically formed using a method that includes the step of disposing the composite (10) within the one or more containers (e.g. the outer container (40)). In various embodiments, the method of forming the packaging system includes the steps of sealing the composite (10) and/or the one or more containers.

### Package for a Medical Device:

In other embodiments, the disclosure provides a package (100) for a medical device (116). The package (100) includes a blister (110). The blister (110) may be as described above or may be different and may be formed by any method described above or by a different method. The blister (110) may be a single layer or may include multiple layers, e.g. any described above.

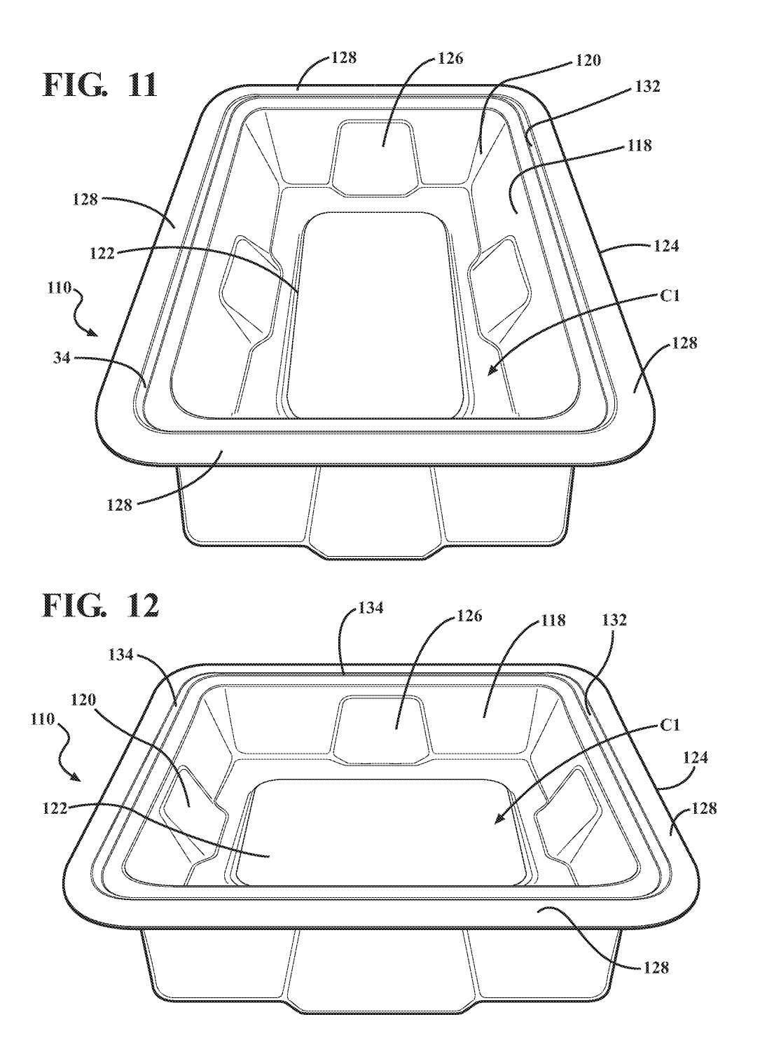

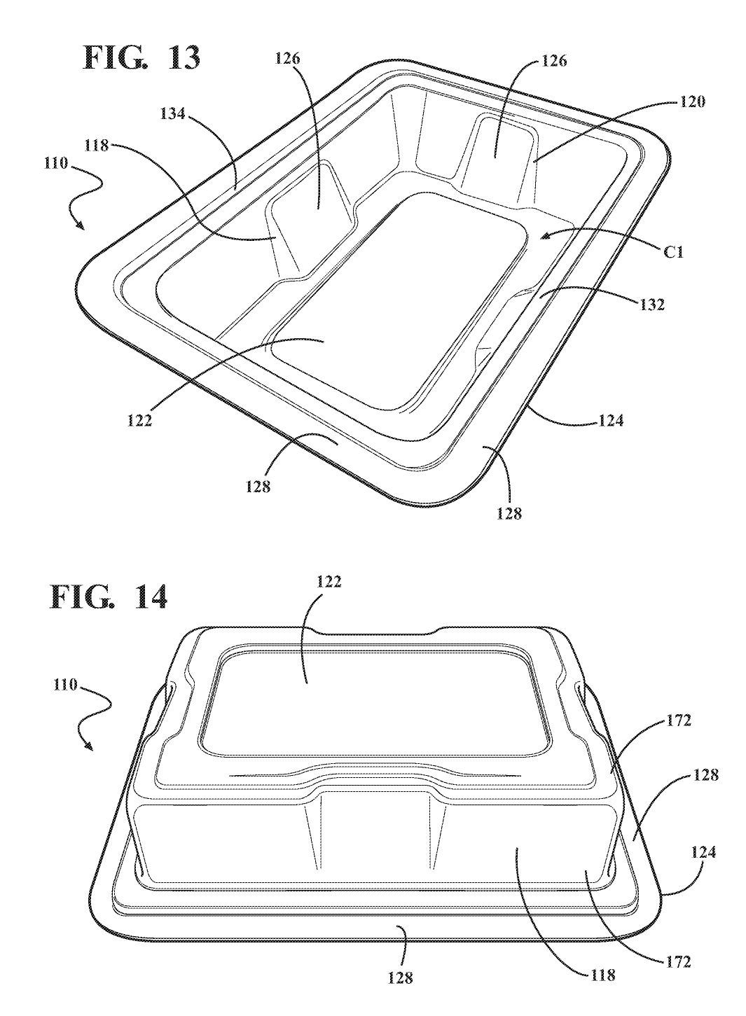

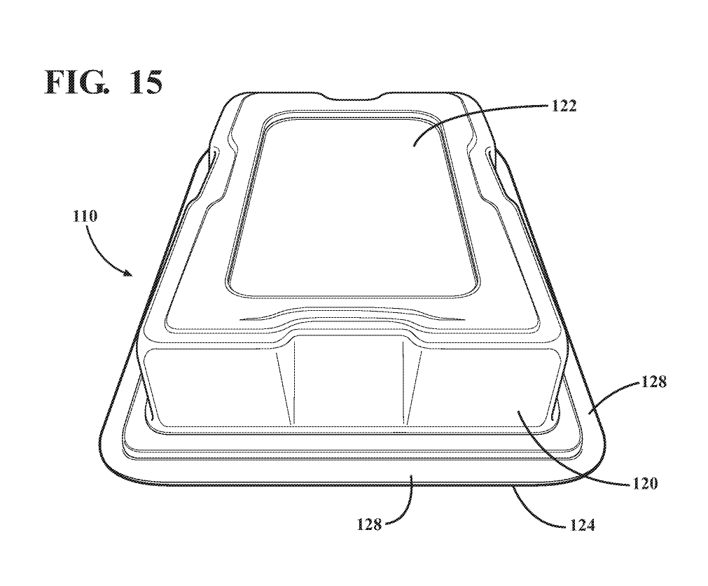

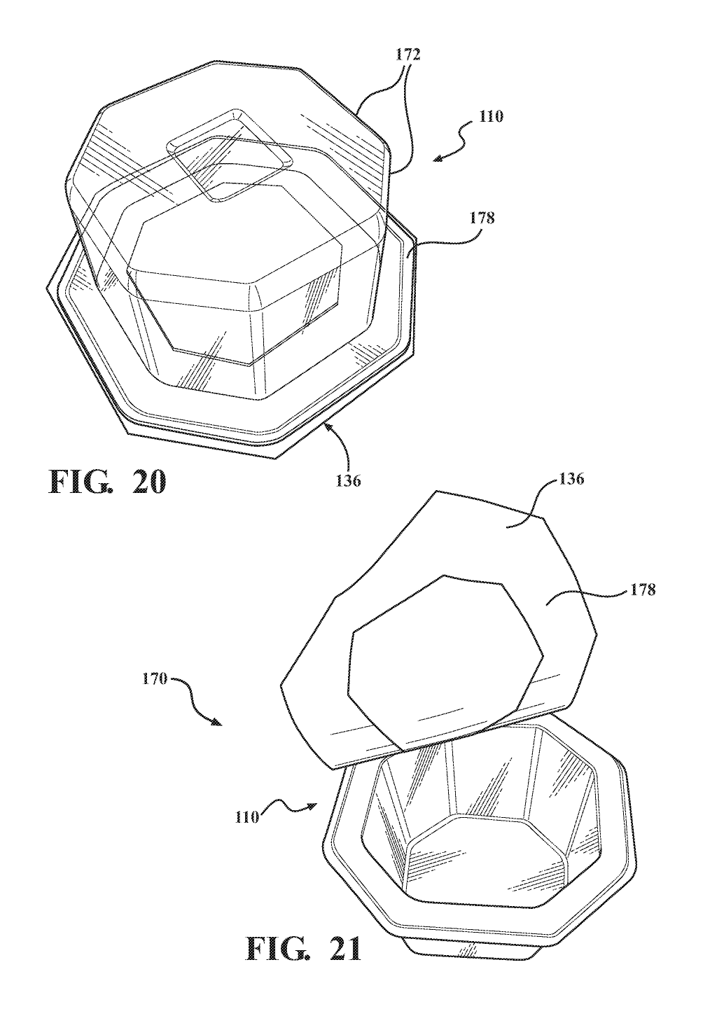

The blister (110) has a base (122) and a plurality of walls (172) extending from the base (122) with the base (122) and the plurality of walls (172) defining a cavity (C1) for receiving the medical device (116), e.g. as shown in FIGS. 11-23 and/or as described above.

The blister (110) has the base (122) having the plurality of walls (172) extending therefrom, typically including side walls (118) and end walls (120), and a periphery (124) (e.g. an edge) of the blister (110), e.g. as shown in FIGS. 11-13. The plurality of walls (172) (e.g. side and/or end walls (118, 120)) may define one or more projections (126) which may engage the medical device (116) to retain its position in the blister (110). Typically, the side walls (118), end walls (120), and base (122) define a rectangular plan form but may define any shape plan form known in the art.

The plurality of walls (172) (e.g. side walls (118) and end walls (120)) and the base (122) typically define the cavity (C1). The cavity (C1) may be open faced or entirely enclosed (i.e., defined on all sides) by the side walls (118), end walls (120), and base (122). The cavity (C1) may be defined as any shape including, but not limited to, cylindrical shapes, spherical shapes, conical shapes, rectangular shapes, cubic shapes, and the like. In one embodiment, the cavity (C1) is defined as a shape that is the same as, substantially similar to, or complementary to, the shape of the medical device (116). In another embodiment, the cavity (C1) is defined as a pocket. Typically the cavity receives the medical device.

The plurality of walls (172) (e.g. side walls (118) and/or end walls (120)) may define top portions ending in flat or planar flanges (128) which are substantially parallel to the base (122). In one embodiment, a stepped recess (132) is formed in one or more of the flanges (128) and is disposed around the cavity (C1). The stepped recess (132) typically includes a recess periphery (e.g. an edge of the stepped recess) (134) that is beveled upward to the flanges (128) which may support a lid (136). In other embodiments, the plurality of walls (172) includes a periphery and at least one flange extending from said plurality of walls (172) and disposed about said periphery.

The package (100) also includes the lid (136) disposed on the blister (110) and at least partially covering the cavity (C1). The lid (136) may also be as described above or may be different. The lid (136) has outer and inner surfaces (130, 178) and includes a high density polyethylene. The high density polyethylene may be any described or known in the art, such a Tyvek. Alternatively, the high density polyethylene may be any as described above.

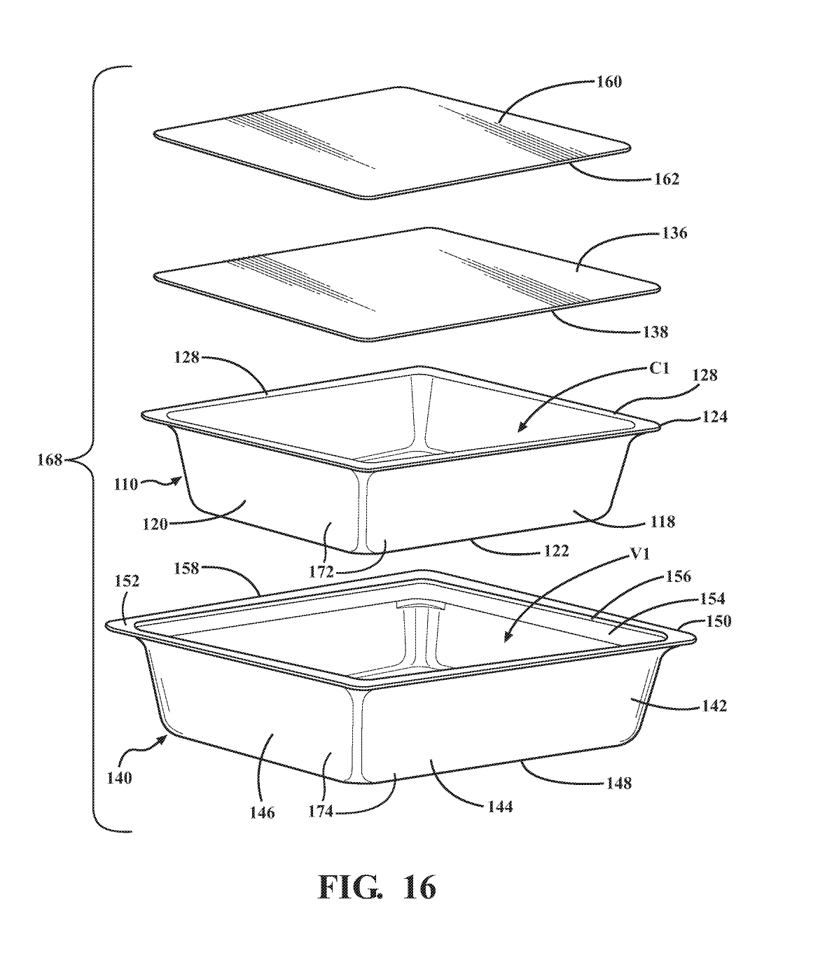

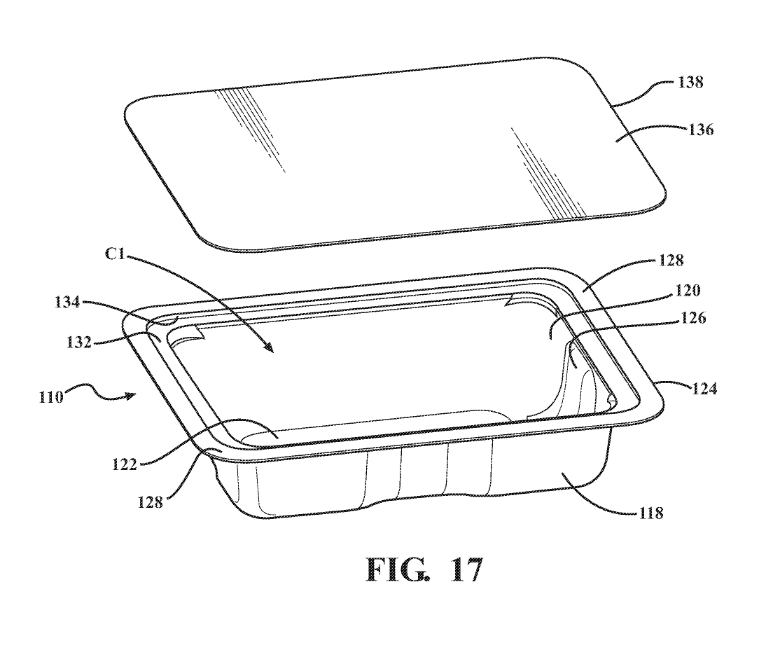

The lid (136) typically has a peripheral edge (138) and is typically disposed on the flanges (128), as shown in FIGS. 16 and 17. The lid (136) may be sized and configured to cover the recess periphery (134) and the stepped recess (132) and the cavity (C1) either in part or in their entirety. In one embodiment, the lid (136) is disposed such that the peripheral edge of the lid (138) aligns with the periphery (124) of the blister (110). In another embodiment, the lid (136) is disposed such that the peripheral edge of the lid (138) does not align with the periphery (124) of the blister (110). In this embodiment, the lid (136) is typically larger than or extends beyond the periphery (124) of the blister (110). The lid (136) is typically formed a suitable material in the art that can withstand sterilization with heat, chemicals, and/or radiation (e.g. ethylene oxide, gamma, e-beam, and/or peroxides) such as any of those described above, such as Tyvek. The lid (136) typically has a thickness of from 1 to 60 mils but is not limited to this thickness. The lid (136) may have any thickness as chosen by one of skill in the art. For example, the lid (136) may have a thickness that is any value or range of values including or between those set forth above. The lid (136) may also be of any color, may be clear, opaque, or transparent. The lid (136) may also be smooth or rough or have any texture known in the art. The lid (136) is typically adhered to the flanges (128) through use of an adhesive. The adhesive may be any known in the art and is not particularly limited. The adhesive may also have any thickness, as chosen by one of skill in the art.

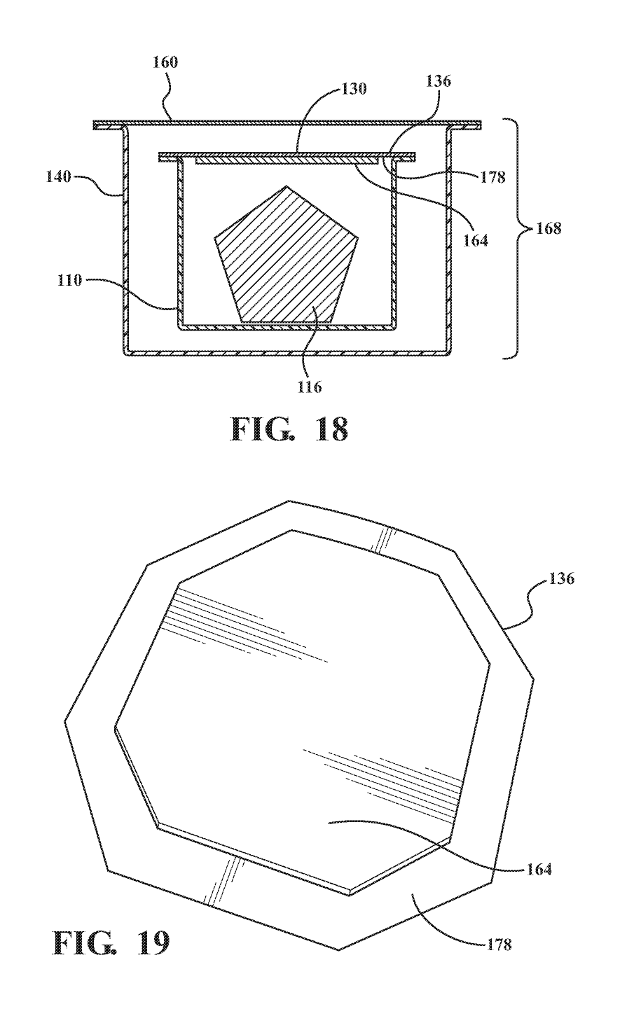

In various embodiments, the blister (110) itself, or one or more layers thereof, includes, is, consists essentially of, or consists of, a first thermoplastic polyurethane or a combination of the first thermoplastic polyurethane and polyethylene terephthalate glycol. The terminology “consists essentially of” describes various embodiments that are free of one or more polymers that are not the first thermoplastic polyurethane or polyethylene terephthalate glycol, and/or free of one of more additives described herein, etc. In various embodiments, the blister (110) is either the first thermoplastic polyurethane or polyethylene terephthalate glycol alone or a co-extrusion of polyethylene terephthalate glycol/first thermoplastic polyurethane with the first thermoplastic polyurethane being an innermost layer of the blister (110). In still other embodiments, the first thermoplastic polyurethane and the polyethylene terephthalate glycol form a single layer (e.g. by co-extrusion). A non-limiting example of such a single layer is set forth in FIG. 18.

The first thermoplastic polyurethane is typically an aromatic polyether polyurethane, such as any described above. In various embodiments, the first thermoplastic polyurethane is commercially available from Covestro under the tradename of Dureflex, e.g. Dureflex X1740E and/or from Bayer Material Science under the tradename of Dureflex X1742E. The first thermoplastic polyurethane may alternatively be any USP Class 6 thermoplastic polyurethane. In various embodiments, the first thermoplastic polyurethane may have one or more of the following physical properties or may have none of the following physical properties or physical properties different from those set forth below.

| | | Range of |
|---|---|---|
| Physical Property | ASTM Test Method | Values* and Units |
| Specific Gravity | D-792 | 1.1-1.5 |
| | | 1.2-1.4 |
| | | 1.2-1.3 |
| Durometer (Shore A) | D-2240 | 80-100 |
| | | 85-95 |
| | | 85-90 |
| Taber Abrasion | D-3489 | 1-50 mg loss |
| (H-18, 1000 g load | | 10-50 mg loss |
| cycles) | | 15-35 mg loss |
| | | 20-30 mg loss |
| Ultimate Tensile Strength | D-882 | 8,000-12,000 psi |
| | | 9,000-11,000 psi |
| | | 9,500-10,000 psi |
| Ultimate Elongation | D-882 | 100-700% |
| | | 400-600% |
| | | 550-600% |
| 100% Modulus | D-882 | 500-1,500 psi |
| (MD/CD Ave.) | | 1,000-1,300 psi |
| | | 1,150-1,250 psi |
| 300% Modulus | D-882 | 2,000-4,000 psi |
| (MD/CD Ave.) | | 2,500-3,500 psi |
| | | 2,700-3,200 psi |
| Min. Softening Point | E2347-04 | 145-160° C. |
| (TMA Onset) | | 300-310° F. |
| | | 150-155° C. |
| | | 300-305° F. |
| Max. Softening Point | E2347-04 | 160-180° C. |
| (TMA Endpoint) | | 330-350° F. |
| | | 170-175° C. |
| | | 335-345° F. |
| Tear Resistance | D-1004 | 400-600 pli |
| (MD/CD Ave.) | | 450-550 pli |
| | | 475-535 pli |

*In various non-limiting embodiments, all values and ranges of values including and between any of those set forth above are expressly contemplated and may be used herein.

The package (170) further includes an insert (164) disposed on and in direct contact with the inner surface (178) of the lid and facing the cavity (C1), wherein the insert (164) includes a second thermoplastic polyurethane for increasing puncture resistance of the lid (136) from the medical device (116).

In various embodiments, the insert (164) is, includes, consists essentially of, or consists of, the second thermoplastic polyurethane. The terminology “consists essentially of” describes various embodiments that are free of one or more polymers that are not the second thermoplastic polyurethane, and/or free of one of more additives described herein, etc. Various non-limiting embodiments are set forth in FIGS. 19, 20, and 21. The second thermoplastic polyurethane may be the same as the first thermoplastic polyurethane or may be different. The second thermoplastic polyurethane may alternatively be any USP Class 6 thermoplastic polyurethane. The second thermoplastic polyurethane may be any thermoplastic polyurethane described above. Typically, the second thermoplastic polyurethane is an aromatic polyether polyurethane, such as any described above. Alternatively, the first and/or second thermoplastic polyurethanes may be a combination (e.g. coextruded combination) of a thermoplastic polyurethane and PETG.

In various embodiments, the blister (110) consists essentially of the first thermoplastic polyurethane or the combination of the first thermoplastic polyurethane and the polyethylene terephthalate glycol. In other embodiments, the insert (164) consists essentially of the second thermoplastic polyurethane. In further embodiments, the lid (136) consists essentially of the high density polyethylene. In still further embodiments, the blister (110) consists of the first thermoplastic polyurethane or the combination of the first thermoplastic polyurethane and the polyethylene terephthalate glycol and the first thermoplastic polyurethane is further defined as an aromatic polyether polyurethane, such as any described above. In other embodiments, the insert (164) consists of the second thermoplastic polyurethane and the second thermoplastic polyurethane is further defined as an aromatic polyether polyurethane, such as any described above. In still other embodiments, the lid (136) consists of the high density polyethylene.

In various embodiments, the inner surface (178) of the lid (136) has a surface area and the insert (164) is disposed about at least 25, at least 30, at least 35, at least 40, etc. percent of the surface area of the inner surface (178). In another embodiment, the inner surface (178) of the lid (136) has a surface area and the insert (164) is disposed about less than 100 percent of the surface area of the inner surface (178). In further embodiments, the insert (164) is disposed about 1, 2, 3, 4, 5, 10, 15, 20, 25, 30, 35, 40, 45, 50, 55, 60, 65, 70, 75, 80, 85, 90, 95, 96, 97, 98, 99, or about 100 percent of the surface area of the inner surface (178). Moreover, in additional embodiments, the insert (164) may be disposed about “at least” one or more of the aforementioned values. The insert (164) is not limited to any particular size and thickness. In various embodiments, the insert (164) has a thickness of from 2 to 40 mils, from 2 to 30 mils, from 2 to 20 mils, or from 2 to 10 mils. Moreover, all values and ranges of values including and between those described above are hereby expressly contemplated in various non-limiting embodiments.

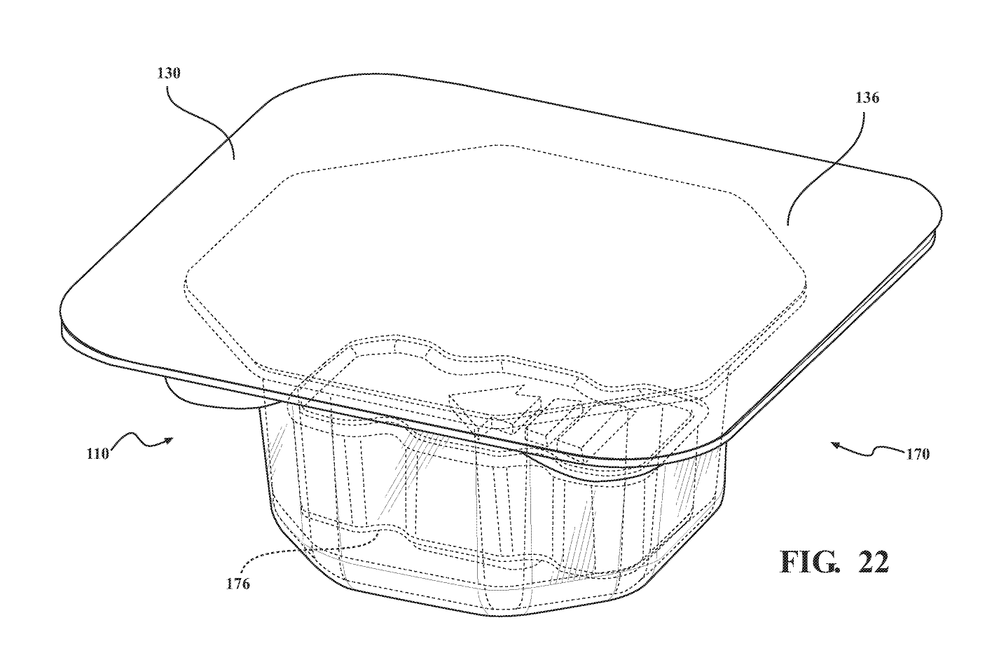

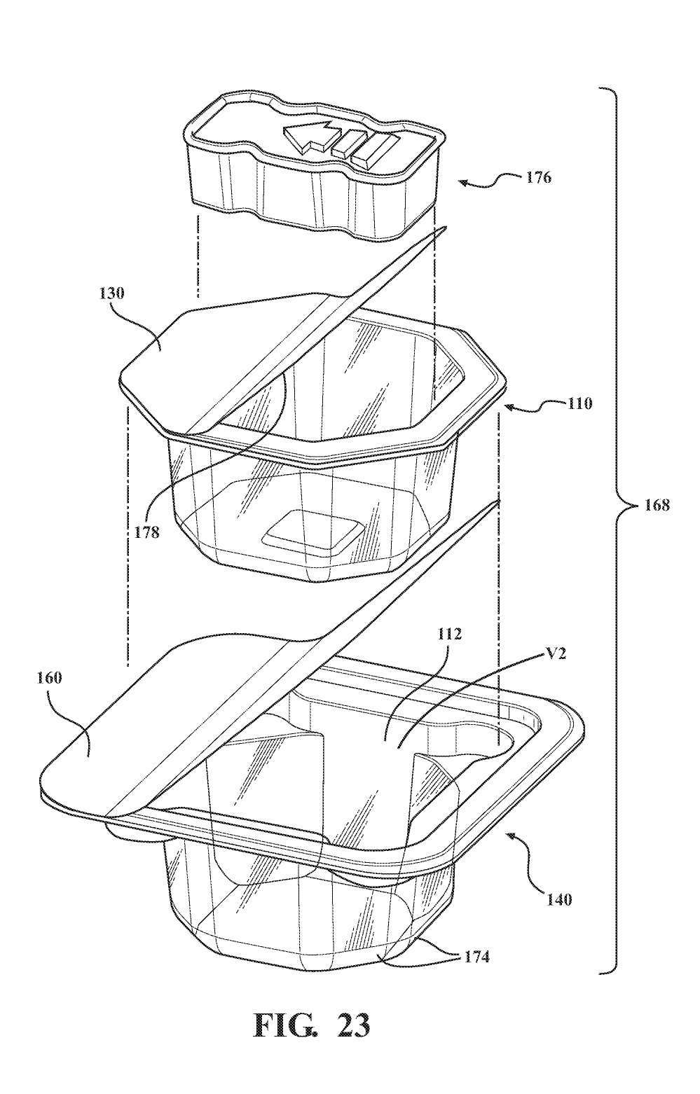

In various embodiments, such as set forth in FIGS. 22 and 23, the package (170) also includes a clamshell (176) or similar enclosure or pouch to house the medical device (116). The clamshell (176) may be, include, consist essentially of, or consist of, any plastic described above, any thermoplastic polyurethane described above, any polymer described above, or any combination thereof. The clamshell may be alternatively described as a protective cover that is not limited in composition, shape, or design, such as the protective cover described above. In one embodiment, the protective cover is further defined as a bag (e.g. a thermoplastic polyurethane bag) and the medical device (116) is placed inside of the bag. Alternatively, the protective cover may be further defined as a three dimensional foam in which the medical device (116) may be placed. In one embodiment, both a bag and a three dimensional foam are utilized. The medical device (116) and the protective cover may then be disposed within the package (170).

This disclosure also provides a method of forming the package (170) wherein the method includes the steps of providing the blister (110); providing the lid (136); disposing the insert (164) on and in direct contact with the inner surface (178) of the lid (136); and disposing the lid (136) on the blister (110), to form the package (170).

### Packaging System:

This disclosure also provides a packaging system (168) for packaging the medical device (116) in a double sterile barrier, e.g. a blister within a blister. Various non-limiting embodiments are set forth in FIGS. 16, 18, and 23. The packaging system (168) includes a container (140) including polyethylene terephthalate glycol. In one embodiment, the container (140) consists essentially of polyethylene terephthalate glycol. In another embodiment, the container (140) consists of polyethylene terephthalate glycol. The terminology “consists essentially of” describes various embodiments that are free of one or more polymers that are not the polyethylene terephthalate glycol, and/or free of one of more additives described herein, etc.

The container (140) has a bottom (148) (or floor) and a plurality of walls (174) (or sides) extending from the bottom (148) to define a void (V2). The packaging system (168) also includes a cover (160) disposed on the container (140) for at least partially covering the void (V2) and comprising a high density polyethylene. The packaging system (168) further includes the aforementioned blister (110) disposed within the void (V2) of the container (140).

The container (140) may be further defined as a film, box, carton, bag, envelope, drum, can, bottle, or the like, or as any described above. The container (140) may be identical in shape (e.g. complementary in shape) to the blister (110).

Typically, the container (140) surrounds the blister (110). In other words, the blister (110) is disposed within the container. Said differently, the container (140) houses the blister (110). In this embodiment, the container (140) is exposed to the environment. In one embodiment, the container (140) is further defined as a pouch with one open, but sealable, end. The open end may be sealed by any means known in the art including physical and/or chemical means. In one embodiment, the end is sealed to create a sterile barrier. In another embodiment, the end is not sealed.

The container (140) typically includes a body (142) that is integrally formed with a plurality of container walls (174), including sides (144) and ends (146), and the floor (148), which together define the void (V2) in which the blister (110) may be disposed. The sides (144) and/or ends (146) may define top portions (150) ending in flat or planar surfaces (152) that are substantially parallel to the floor (148). In one embodiment, a stepped indentation (154) is formed in one or more of the surfaces and is disposed around the void (V2). The stepped indentation (154) typically includes an edge (156) that is beveled upward to the flat or planar surfaces (152). The container (140) also defines a periphery (158).

The cover (160) typically includes a peripheral edge (162). Typically, the cover (160) is disposed such that the peripheral edge of the cover (162) aligns with a periphery of the container (140). The cover (160) typically has a thickness of from 1 to 60 mils but is not limited to this thickness. The cover (160) may have any thickness as chosen by one of skill in the art. The cover (160) may also be of any color, may be clear, opaque, or transparent. The cover (160) may also be smooth or rough or have any texture known in the art.

Both the container (140) and the packaging system (168) itself, may be formed by any method or means known in the art. Typically, the container (140) is formed using any one or more processes described above, e.g. using thermoforming and/or lamination techniques. In various embodiments, the packaging system (168) is typically formed using a method that includes the step of disposing the blister (110) within the container (140). In various embodiments, the method of forming the packaging system (168) includes the steps of sealing the blister (110) and the container (140).

It is to be appreciated that all values and ranges of values between and including those set forth above are hereby expressly contemplated for use in various non-limiting embodiments. Moreover, any one or more components, compounds, method steps, etc. described above may be used with any one or more other components, compounds, method steps, etc. described above, in various non-limiting embodiments, even if such a combination is not described in the same paragraph or section.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described in an illustrative manner, and it is to be understood that the terminology which has been used is intended to be in the nature of words of description rather than of limitation. Obviously, many modifications and variations of the present invention are possible in light of the above teachings. It is, therefore, to be understood that within the scope of the appended claims, the present invention may be practiced otherwise than as specifically described.

What is claimed is:

1. A package for a medical device, wherein said package comprises:
a blister having a base and a plurality of walls extending from said base with said base and said walls defining a cavity for receiving the medical device, said blister comprising a first thermoplastic polyurethane or a combination of said first thermoplastic polyurethane and polyethylene terephthalate glycol;
a lid disposed on said blister and at least partially covering said cavity, wherein said lid has outer and inner surfaces and comprises a high density polyethylene; and
an insert disposed on and in direct contact with said inner surface of said lid and facing said cavity, wherein said insert comprises a second thermoplastic polyurethane for increasing puncture resistance of said lid from the medical device.

2. The package of claim 1 wherein said inner surface of said lid has a surface area and insert is disposed about at least 25 percent of said surface area of said inner surface.

3. The package of claim 1 wherein said inner surface of said lid has a surface area and insert is disposed about less than 100 percent of said surface area of said inner surface.

4. The package of claim 1 wherein said insert has a thickness of from 2 to 40 mils.

5. The package of claim 1 wherein said first thermoplastic polyurethane is further defined as an aromatic polyether polyurethane.

6. The package of claim 5 wherein said second thermoplastic polyurethane is further defined as an aromatic polyether polyurethane.

7. The package of claim 1 wherein said blister consists essentially of said first thermoplastic polyurethane or said combination of said first thermoplastic polyurethane and said polyethylene terephthalate glycol.

8. The package of claim 7 wherein said insert consists essentially of said second thermoplastic polyurethane.

9. The package of claim 8 wherein said lid consists essentially of said high density polyethylene.

10. The package of claim 1 wherein said blister consists of said first thermoplastic polyurethane or said combination of said first thermoplastic polyurethane and said polyethylene terephthalate glycol and said first thermoplastic polyurethane is further defined as an aromatic polyether polyurethane

11. The package of claim 10 wherein said insert consists of said second thermoplastic polyurethane and said second thermoplastic polyurethane is further defined as an aromatic polyether polyurethane

12. The package of claim 11 wherein said lid consists of said high density polyethylene.

13. The package of claim 1 wherein said plurality of walls include one or more projections for engaging the medical device.

14. The package of claim 1 wherein said plurality of walls include a periphery and at least one flange extending from said plurality of walls and disposed about said periphery.

15. A method of forming the package of claim 1 comprising the steps of:
providing the blister;
providing the lid;
disposing the insert on and in direct contact with the inner surface of the lid; and
disposing the lid on the blister to form the package.

16. A packaging system for packaging a medical device in a double sterile barrier, wherein said packaging system comprises:
a container comprising polyethylene terephthalate glycol and having a bottom and a plurality of walls extending from said bottom to define a void;
a cover disposed on said container for at least partially covering said void and comprising a high density polyethylene;
a blister disposed within said void of said container, wherein said blister has a base and a plurality of walls extending from said base with said base and said walls defining a cavity for receiving the medical device, said blister comprising a first thermoplastic polyurethane or a combination of said first thermoplastic polyurethane and polyethylene terephthalate glycol;
a lid disposed on said blister and at least partially covering said cavity, wherein said lid has outer and inner surfaces and comprises a high density polyethylene; and
an insert disposed on and in direct contact with said inner surface of said lid and facing said cavity, wherein said insert comprises a second thermoplastic polyurethane for increasing puncture resistance of said lid from the medical device.

17. The packaging system of claim 16 wherein said container consists essentially of polyethylene terephthalate glycol.

18. The packaging system of claim 17 wherein said container consists of polyethylene terephthalate glycol.
